(19) United States
(12) Patent Application Publication
(10) Pub. No.: US 2015/0182455 A1
(43) Pub. Date: Jul. 2, 2015

(54) Cannabinoids alcohol mixtures, methods to make and use of the same

(71) Applicant: Michael R. Llamas, San Diego, CA (US)

(72) Inventor: Michael R. Llamas, San Diego, CA (US)

(73) Assignee: HDDC Holdings, LLC

(21) Appl. No.: 14/517,658

(22) Filed: Oct. 17, 2014

Related U.S. Application Data
(60) Provisional application No. 61/893,334, filed on Oct. 21, 2013.

(51) Int. Cl. A61K 9/00; C12G 3/04; C12G 1/022; A61K 31/047; C12C 5/02

(52) U.S. Cl. CPC A61K 9/0095; A61K 31/047; C12C 5/02; C12G 1/0203; C12G 3/04

(57) ABSTRACT

The present invention relates to a composition comprising about 0.001% to about 1% by weight of cannabinoids or derivatives thereof, based on the total weight of the mixture, and an alcohol, with water and other compositions as the balance of the weight. The present invention also relates to an alcoholic beverage containing between about 0.001% to about 1% (wt/wt) cannabinoids and derivatives thereof, between about 2% to about 45% (wt/wt) of alcohol, with water, fermentation byproducts, emulsifiers, and flavor enhancers composing the balance of the total weight. The invention also discloses methods to prepare these compositions, and methods of use to minimize adverse effects from alcohol consumption.

### CROSS REFERENCE TO RELATED APPLICATIONS

This application claims the benefit under 35 U.S.C. §119(e) of U.S. Provisional Application No. 61/893,334, filed Oct. 21, 2013.

### FIELD OF THE INVENTION

Described herein are compositions of cannabinoids and derivatives thereof with alcohol, water, other fermentation by-products, emulsifiers, and flavor enhancers, to be consumed orally to minimize the adverse effects of alcohol consumption.

### BACKGROUND OF THE INVENTION

Cannabis has long been used both for medicinal and recreational purposes. The active ingredients of cannabis include cannabinoids and Δ⁹-tetrahydrocannabinol (THC). Among the cannabinoids, cannabidiol (“CBD”) is well-known as lacking the psychoactive effect that Δ⁹-tetrahydrocannabinol has. CBD was shown in laboratory settings to be clinically useful to reduce inflammation, help alleviate nausea and emesis, treat epilepsy, anxiety disorders, or glaucoma. CBD can also provide analgesia effects and neuro-protection.

The IUPAC nomenclature of THC is (−)-(6aR,10aR)-6,6,9-trimethyl-3-pentyl-6a,7,8,10a-tetrahydro-6H-benzo[c]chromen-1-ol. Upon consumption by a human subject, THC binds to the cannabinoid receptor CB₁, which is located in brain tissues. Cannabidiol's IUPAC nomenclature is 2-((1S,6S)-3-methyl-6-(prop-1-en-2-yl)cyclo-hex-2-enyl)-5-pentylbenzene-1,3-diol). Different from THC, CBD does not bind to CB₁ or CB₂, two different endocannabinoid receptors. Cannabichromene (CBC) has the IUPAC nomenclature of 2-methyl-2-(4-methylpent-3-enyl)-7pentyl-5-chromenol. CBC is shown to have anti-inflammatory and anti-viral effects, as well as analgesic effects.

The synthetic cannabinoid nabilone (racemic(6aR,10aR)-1-hydroxy-6,6-dimethyl-3-(2-methyloctan-2-yl)-7,8,10,10a-tetrahydro-6H-benzo[c]chromen-9(6aH)-one) is believed to have fewer undesired side effects than THC. Nabilone is used for antiemetic and analgesic purposes for neuropathic pain. The U.S. Food and Drug Administration approved nabilone for treatment of chemotherapy-induced nausea and vomiting.

Synthetic THC, e.g. dronabinol ((−)-(6aR,10aR)-6,6,9-trimythel-3-pentyl-6a,7,8,10a-tetrahydro-6H-benzo[c]chromen-1-ol)), is also sold on the market under the name Marinol®. Marinol® tablets dissolve in the mouth upon oral administration. However, the tablet must be kept under the tongue for the period that it takes to dissolve the tablet. Synthetic THC is used to treat a variety of conditions, including lack of appetite in Acquired Immune Deficiency Syndrome (AIDS) patients, severe nausea, and vomiting.

Cannabinoids are also consumed by other methods, including inhalation, either directly by burning of the cannabis plant materials, or indirectly by vaporization of the plant material and cooling of the resulting vapor for inhalation. The consumption of cannabinoids by inhalation has many disadvantages, including exposure to harmful substances by the respiratory system. Many people dislike smoking in general; and the residual smell of the cannabis plant material after burning is also undesirable. Controlling the dosage in smoking of the plant material is a challenging problem. Vaporization is an alternative that can be controlled in dosage. However, the convenience of setting up a vaporizer is a challenge for many. Also, these methods of consumption do not address the problems raised below.

Alcohol has been long used as a beverage and food additive. Sale of alcohol is popular in the United States as well as the entire world. The use of alcohol for food and beverage purposes is a widespread practice. On the other hand, alcohol consumption also poses many problems to consumers. Among the problems is the “hangover” effect, characterized by thirst, headaches, lethargy, muscle aches, nausea, vomiting, stomach pain, poor or decreased sleep, increased sensitivity to light and sound, among other symptoms. Hangover symptoms affect an individual's ability to concentrate and work effectively.

A hangover is caused by the dehydration and decreased blood sugar level associated with alcohol consumption. Dehydration is the cause of thirst, headache, nausea, and vomiting. Alcohol also causes inflammation of the stomach lining, which causes diarrhea, which further contributes to dehydration. Over the time, hangovers will alleviate when the decreased level of blood sugar increases back to a normal level and dehydration decreases.

Alcohol is metabolized in two stages: ethanol is broken down to acetaldehyde, which is a toxin; acetaldehyde is broken down to acetate, which is the active ingredient in household vinegar. Acetaldehyde causes rapid pulse, sweating, skin flushing, nausea, and vomiting in human subjects. Inability to digest acetaldehyde quickly is a documented phenomenon among certain ethnic groups, including East Asians and Native Americans. According to the National Institutes of Health, East Asians and Native Americans have a higher instance of a gene that causes inactivity of the enzyme aldehyde dehydrogenase-2, which metabolizes acetaldehyde into acetate. Glutathione is the other enzyme that metabolizes acetaldehyde. Acetaldehyde also contributes to hangover symptoms.

Alcohol consumption also causes liver diseases such as fatty liver disease, inflammation in the liver (alcohol hepatitis), scarring, and cirrhosis of the liver. Ethanol, the main active ingredient in alcoholic beverages, damages the liver by being digested into acetaldehyde, which is a toxin. Alcoholic fatty liver disease can also result from heavy drinking. The National Health Service (United Kingdom) estimates that between 90% to 100% of heavy drinkers are affected by alcoholic fatty liver disease.

Fatty liver disease occurs after heavy consumption of alcohol. Extra fat is built up in liver cells in fatty liver disease. While most fatty liver diseases are asymptomatic, symptoms may include fatigue, weakness, and weight loss. Fatty liver disease is reversible with alcohol abstinence. Usually, a few weeks of alcohol abstinence after an acute drinking episode may be enough for liver re-generation and fatty liver disease to disappear.

Alcoholic hepatitis encompasses a wide range of liver injury, wherein prolonged alcohol consumption causes inflammation of the liver. The spectrum of severity ranges from asymptomatic inflammation to liver failure and possible death. Common symptoms may include loss of appetite, nausea, vomiting, abdominal pain, fever, and jaundice (yellowing of the skin and of the whites of the eyes). The American Liver Foundation estimates that up to 35% of heavy drinkers develop alcoholic hepatitis.

Cirrhosis is the “scarring” of the liver, wherein normal hepatic parenchyma is replaced with thick bands of fibrous tissue. Essentially, tissues affected by cirrhosis become hard instead of soft and healthy. When the liver has extensive cirrhosis, portal hypertension and liver failure may result. Cirrhosis is irreversible. At this stage of liver damage, abstinence from alcohol can prevent further damage, but cannot reverse the damage already done.

The National Institute on Alcohol Abuse and Alcoholism estimates that approximately 61% of women and 72% of men aged 18 and above drank at least once in the past year. Alcohol consumption is a widespread practice among adults. Equally widespread are the adverse effects of alcohol, ranging from mild irritation to a hangover to long term effects. Most drinkers accept these adverse effects as part of the alcohol consumption experience.

Nevertheless, alcohol consumption will continue, both as a food and a beverage product. While drinking in moderation is recommended to alleviate some of the problems and diseases as outlined above, there is still a need to offset the hangover effect and other long-term effects of alcohol consumption. An alcoholic beverage or an alcoholic food additive that can be consumed with minimal or lessened adverse effects on the human body is highly desirable.

Additionally, as a food and beverage, an alcohol mixture must maintain its normal beverage taste and diverse flavors for marketing purposes. Many additives are used in the fermentation industry to enhance alcoholic beverage flavors, such as mint, orange, lime, cookie dough, chocolate, jalapeno, cinnamon, marshmallow, coffee, etc. to enhance the flavor and tasting experience. Solving the alcohol adverse effect problem should not affect alcoholic beverage products' ability to deliver a desirable drinking experience.

### Abbreviations

CBC: Cannabichromene

CBD: Cannabiodiol

CO₂: Carbon dioxide

IUPAC: International Union of Pure and Applied Chemistry

THC: Tetrahydrocannabinols

v/v: volume/volume

wt/wt: weight/weight

### SUMMARY OF THE INVENTION

The present invention relates to a composition of cannabinoids and/or derivatives thereof with an alcohol, and other ingredients, such as water, carbohydrates, or flavor enhancers. The present invention also relates to the making of this alcohol and cannabinoid composition. The present invention further relates to the use of this composition for a mammal's consumption, preferably a human, to prevent or reduce alcohol's adverse effects on humans and mammals, while retaining the taste experience and psychological effect commonly associated with alcoholic beverage consumption.

### DETAILED DESCRIPTION OF THE INVENTION

This present invention is capable of being embodied in various forms. The description below of several embodiments is made with the understanding that the present disclosure is to be considered as an exemplification of the claimed subject matter, and is not intended to limit the attached claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the verb “to comprise” in this description, claims, and other conjugations are used in its non-limiting sense to mean that items following the word are included, but items not specifically mentioned are not excluded.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more.”

The word “alcohol” used in this description, claims, and other conjugations are used to mean ethanol (CH₃—CH₂—OH), or a mixture having ethanol as the primary component and the balance is water, carbohydrates, flavor enhancer, or other components commonly found in alcoholic beverages, or other organic alcohol compounds having the functional group (—OH), or a mixture of other organic alcohol compounds with other components as necessary.

The word “cannabinoid” used in this description, claims, and other conjugations is used to mean any compound that interacts with a cannabinoid receptor and other cannabinoid mimetics, including, but not limited to, certain tetrahydropyran analogs (Δ⁹-tetrahydrocannabinol, Δ⁸-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1hydroxy-6,6-dimythel-9H-dibezo[b,d]pyran-9-ol,(−)-(3S,4S)-7-hydroxy-delta-6-tetrahydrocannabinol-1,1-dimethylheptyl, (+)-(3S,4S)-7-hydroxy-Δ-6-tetrahydrocannabinol, and Δ⁸-tetrahydrocannabinol-11-oic acid); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)); certain aminoalkylindole analogs (e.g., (R)-(+)-[2,3-dihydro-5-methyl-3-(4-morpholinylm-ethyl)-pyrrolo[1,2,3,-de]-1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1,3-benzendiol, and 4-(1,1-dimethylheptyl)-2,3′-dihydroxy-6′-α-(3-hydroxypropyl)-1′,-2′,3′,4′,5′,6′-hexahydrobiphenyl); their salts, solvates, metabolites, and metabolic precursors.

The word “cannabidiol” refers to cannabidiol, cannabidiol prodrugs, and cannabidiol derivatives.

This present invention relates to a composition comprising cannabinoids and derivatives thereof with an alcohol and other components, thereby providing a composition that can be consumed orally or topically with lessened adverse effects commonly associated with alcohol consumption. A mammal, preferably a human, by consuming this mixture, is free or alleviated from adverse effects of alcohol consumption, while achieving the same tasting, physical, and psychological effects of alcohol consumption.

According to the present invention, the composition preferably comprises a wide range of cannabinoids and derivatives thereof in weight percentage. The balance in weight of the mixture is alcohol and water, as appropriate. Different weight percentages of cannabinoids and derivatives thereof in combination with different weight percentages of alcohol may give different beneficial effects of the cannabinoids.

The cannabinoids or derivatives thereof may be in solid form at room temperature, which contains a certain amount of cannabinoids or derivatives thereof. Liquid form cannabidiol can be obtained from the extracts of natural sources and are readily available on the legal market if the products are considered hemp finished products. Natural sources include plants from the cannabis genus, such as Cannabis sativa, Cannabis ruderalis, or Cannabis indicia.

Alternatively, cannabinoids and derivatives thereof can be obtained from synthetic sources. The synthesis of THC can be found in U.S. Pat. No. 7,186,850 B2. The synthesis of cannabidiol can be found in Navak & Salemink, Tetrahedron Lett. 23, 253 (1982). These publications are included herein as references.

Synthetic cannabinoids and derivatives thereof used in these embodiments are substantially free of impurities. “Substantially free of impurities” as used herein means that impurities are present in the amount by weight of the composition of less than about 30%, less than about 20%, less than about 15%, less than about 10%, less than about 5%, less than about 2%, less than about 1%, or less than about 0.1%.

Naturally cannabinoids and derivatives thereof may be provided in oily form, typically known as hemp oil or cannabis oil. When provided in this form, hemp oil or cannabis oil may contain various cannabinoids at the amount by weight between about 5% to about 50%. Other components of hemp oil or cannabis oil may be vegetable triglycerides, fatty acids, such as linoleic acid and α-linoleic acid, as well as other impurities such as β-caryophyllene, myrcene, and β-sitosterol.

Certain crystallized dissolvable cannabinoid complexes are also available and may be used in these embodiments. Hemp oil or cannabis oil containing naturally occurring cannabinoids may be distilled to remove fatty acid content. Crystallized cannabinoids are then complexed with other matters to increase solubility. Alternatively, crystallized cannabinoids may also be added directly into the alcoholic drink for consumption.

Methods to complex cannabinoids are disclosed in various prior arts. Viemstein et. al. Patent Application Publication US2013/0295026 describes the complexing of synthetic Δ⁹-tetrahydrocannabinols with randomly methylated β-cyclodextrin to increase solubility in water. Jarho et. al. U.S. Pat. No. 7,592,328 discloses complexing cannabinoids with cyclodextrin, such as α-cyclodextrin, β-cyclodextrin, and γ-cyclodextrin. Other methods to complex cannabinoid(s) with other agents are also available.

In this alcohol mixture, the cannabinoids and derivatives thereof concentration is preferably at about 0.001% to about 1% (wt/wt), or preferably at about 0.005% to about 0.01% (wt/wt), preferably at about 0.01% to about 0.07% (wt/wt), and preferably about 0.13% to about 0.18% (wt/wt). These concentration ranges are useful in providing relief for hangover symptoms.

The alcohol in this embodiment may be made from industrially available methods or from traditional fermentation methods. The kind of the alcohol beverage used may be selected based on need. The presence of fermentation by-products depends on the nature of the alcoholic beverage used. A composition according to this embodiment may also be consumed orally, if appropriate, by mammals, preferably a human being. Industrially manufactured alcohol must not be denatured.

In another preferred embodiment, the alcohol in this composition is ethanol. Preferably, the cannabinoid and alcohol composition is consumed orally as a beverage or food. The alcohol composition therefore further comprises water, carbohydrates, flavor enhancers, food color additives, preservatives, and other fermentation byproducts. The alcohol percentage and the production method define its product identity, such as beer, wine, distilled liquor, other alcoholic beverages, or food. Accordingly, it is preferred that the cannabinoids and derivative thereof percentage ranges from about 0.001% to about 1% by weight, based on the total weight of the alcohol composition. It is also preferred that the cannabinoids and derivatives thereof component does not affect the taste of the alcohol composition as a whole. Cannabinoids and derivatives thereof are tasteless in their pure forms. At this low concentration, cannabinoids and derivatives thereof generally do not affect the taste of the alcohol composition.

The alcohol composition may be produced by traditional fermentation processes. Cannabinoids and derivatives thereof can be added during the fermentation process in appropriate amounts. Typically, cannabinoids and derivatives thereof are premixed with an emulsifier before addition during the fermentation process. Homogenization of the premixed mixture comprising of cannabinoids and derivatives thereof, an emulsifier, and water may be appropriate.

When the alcoholic beverage to be produced is a beer, the production process may comprise the steps of milling the malt, mashing, lautering, boiling, whirlpooling, fermenting, maturing, filtering, and packaging. Stages where cannabinoids and derivatives thereof can be added include the steps of fermenting, maturing, or the final stage prior to packaging.

When the alcoholic beverage to be produced is a wine, the production process may comprise the steps of crushing the grapes, pressing the juice, fermentation, clarification, stabilization, and storage. Stages where cannabinoids and derivatives thereof may be added include the steps of fermentation, clarification, stabilization, or before the storage step.

When the alcoholic beverage to be produced is a distilled spirit, the production process may comprise the steps of mashing, fermenting, washing, distillation, filtering, and packaging. As distilled beverage is distilled, it is recommended that cannabinoids and derivatives thereof are added just before the packaging to ensure the cannabinoid content is present in the alcoholic composition.

Preferably, the cannabinoids and derivatives thereof also disperse in the alcohol composition as a whole. The cannabinoids and derivatives thereof are typically hydrophobic. To ensure dispersion, an emulsifier may be mixed with cannabinoids and derivatives thereof before being added into the composition. Mixing methods using a propeller at high speed may be adequate to ensure the alcohol composition is well dispersed. For a better method of dispersion, homogenization of the cannabinoids and derivatives therefore mixture with an emulsifier in water is recommended.

When cannabinoid complexes are used, emulsifiers may not be needed if the complexes are dissolvable. If the complexes are not dissolvable, emulsifiers are necessary to facilitate the distribution of cannabinoids in the alcoholic composition.

Emulsifiers used in this mixture may be alginic acid, sodium alginate, potassium alginate, ammonium alginate, calcium alginate, propane-1,2-diol alginate, agar, carrageenan, locust bean gum (carob gum), guar gum, tragacanth, gum acacia, xanthan gum, sorbitol, mannitol, glycerol, lecithin, pectin, amidated pectin, sodium and potassium phosphates, sodium and potassium polyphosphates, microcrystalline, methylcellulose, hydroxypropylcellulose, hydroxypropyl-methylcellulose, ethylmethylcellulose, carboxymethylcellulose, sodium, potassium, and calcium salts of fatty acids, mono- and di-glycerides of fatty acids, esters of mono- and di-glycerides of fatty acids, sucrose esters of fatty acids, sucroglycerides, polyglycerol esters of fatty acids, propane-1,2-diol esters of fatty acids, sodium stearoyl-2-lactylate, calcium stearoyl-2-lactylate, and stearyl tartrate, propylene glycol alginate, polyethylene glycol 400, among other emulsifiers. The best emulsifiers for this purpose are gum acacia and quillaia extract.

The amount of emulsifiers used varies dependent on the type of emulsifier. Gum acacia may comprise by weight between about 1% to about 2% of the alcohol composition. Quillaia extract may comprise by weight between about 0.5% to about 1.5% of the alcohol composition.

In a preferred embodiment, the composition comprises of about 0.001% to about 1% wt/wt, about 0.005% to about 0.01% (wt/wt), about 0.001% to about 0.07% (wt/wt), or about 0.13% to about 0.18% of cannabinoids and derivatives thereof, with about 2% to about 45% (wt/wt) of ethanol, with the balance of the composition in weight being water, fermentation by-products, food color additives, preservatives, carbohydrates, etc. The concentration of ethanol in this alcoholic beverage mixture is preferably at about 2% to about 6%, at about 6% to about 15%, or at about 15% to about 45%.

The composition preferably further comprises flavor enhancers, such as mint, cinnamon, cookie dough, chocolate, vanilla, jasmine, lychee, hops, lime, orange, citrus, cucumber, menthol, oak, cherry, or any other flavors to enhance the tasting experience. The weight percentage of the flavor enhancers varies depending on need. The addition of flavor enhancers is only necessary to give the composition the desired flavor. The weight percentage of the flavor enhancer is in the range of about 0.5% to about 10%, more preferably in the range of about 0.5% to about 2%, and even more preferably in the range of about 1% to about 2%.

The composition may further comprise carbon dioxide (CO₂) for flavor enhancement. CO₂ is present in the composition preferably at about 2% to about 3% (v/v), even though other amounts may be effective. CO₂ gives the alcohol composition the typical “gas” taste. CO₂ may be added after the cannabinoids and derivatives thereof have been mixed into the composition.

The composition according to the present invention may further comprise a sweetener, such as sugar alcohols, including xylitol, sorbitol, isomalt, artificial sweeteners, such as aspartame, sucralose, acesulfame potassium, saccharin, or other sweeteners, or natural sweeteners, such as sucrose, maltose, or fructose. The use of sweeteners is optional as the composition can be tailored to the desired taste. The amount of sweetener to be used is from about 0.1% to about 10% by weight.

The composition may be prepared by alcohol fermentation processes known in the art, where cannabinoids and derivatives thereof may be added at different stages and at the end of the fermentation process, where the composition is ready for consumption, bottling, packaging, or further processing. Addition of desired flavor enhancers or sweeteners is accomplished during the fermentation process as needed.

While cannabinoids and derivatives thereof are hydrophobic, they disperse with the use of an emulsifier. When the alcohol composition with cannabinoids and derivatives thereof is consumed orally, the emulsifier used should be a food-grade emulsifier, with an appropriate concentration to ensure safe consumption. An emulsifier may be combined with cannabinoids and derivatives thereof prior to being added into the alcohol composition. The alcohol composition may then be mixed using a propeller at high speed. Homogenization of hemp oil or cannabis oil in the alcoholic drink in the presence of an emulsifier may be required.

This invention also relates to a method to consume an alcohol composition wherein the composition comprises cannabinoids and derivatives thereof to minimize adverse effects of alcohol consumption on a mammal, preferably a human being. An alcohol composition as described in the above preferred embodiment may be consumed orally by a mammal, preferably a human being, wherein the mammal suffers lessened adverse effects from the above alcohol composition consumption.

Adverse effects from alcohol consumption in a mammal, preferably a human being, may be nausea, lethargy, muscle pain, thirst, headache, vomiting, stomach pain, decreased sleep, sensitivity to light and sound, dizziness, rapid heartbeat, red eyes, shakiness, decreased ability to concentrate, mood disturbance, depression, anxiety, irritability, unconsciousness, fatty liver, alcoholic hepatitis, and cirrhosis. By consuming the alcohol composition comprising cannabinoids and derivatives thereof, a mammal, preferably a human being, does not suffer from adverse effects associated with alcohol consumption.

### EXAMPLES

### Example 1

### Alcohol Composition Preparation (Beer)

Ethanol alcohol was used to prepare the alcoholic composition containing 30 mg of CBD.

### Percentages are by Weight

| | Weight | Unit | % w/w | Phase |
|---|---|---|---|---|
| Water | 912.785 | g | 91.263 | A |
| Ethanol | 37 | g | 3.699 | A |
| Carbohydrates | 27 | g | 2.700 | A |
| Nitrogenous compounds | 2.5 | g | 0.250 | A |
| Inorganic | 1.5 | g | 0.150 | A |
| Glycerol | 1.5 | g | 0.150 | A |
| Hop, organic acids | 2.5 | g | 0.250 | A |
| Orange aroma oil | 5 | g | 0.500 | A |
| Other alcohols | 65 | mg | 0.006 | A |
| Organic acids | 100 | mg | 0.010 | A |
| Esters | 20 | mg | 0.002 | A |
| Hemp oil with | 200 | mg | 0.020 | B |
| CBD at 15% | | | | |
| (wt/wt) | | | | |
| Emulsifier | 10 | g | 1.000 | B |
| Total | 1000.17 | g | 100.000 | |

Phase A: an alcohol mixture is obtained from fermentation. The added flavor (orange aroma oil) is mixed into the alcohol mixture for added taste.

Phase B: cannabinoids and derivatives thereof, in this case, cannabidiol, are obtained and mixed with the emulsifier. The cannabinoid-emulsifier mixture is then mixed into the alcohol mixture.

Stirring is recommended, and a propeller at a speed between 20,000 rpm-40,000 rpm can disperse the cannabinoids and derivatives thereof in the body of the alcohol composition.

Hereafter, the alcohol composition with cannabinoids and derivatives thereof with a mass at about 1000 g was prepared.

### Example 2

### Case Studies

An alcohol composition with cannabinoids and derivatives thereof according to Example 1 was consumed by healthy volunteers. Volunteers experienced the same taste as compared to an alcohol composition without cannabinoids during consumption. Volunteers consumed a quantity sufficient to the volunteers to feel “drunk.” In a 12-hour follow up, volunteers reported no hangover symptoms. Volunteers reported no thirst, fatigue, nausea, headache, muscle ache, or other hangover symptoms.

### Example 3

### Alcohol Composition Preparation

| | Weight | Unit | % wt/wt | Phase |
|---|---|---|---|---|
| Ethanol | 13.2 | g | 4.02 | A |
| Water* | 313.6 | g | 95.61 | A |
| Hemp oil with | | | | |
| CBD at 15% | 0.2 | g | 0.06 | B |
| (wt/wt) | | | | |
| Emulsifier (gum | | | | |
| acacia) | 1 | g | 0.30 | B |
| Total | 328 | | 100 | |

Phase A: an alcohol mixture is obtained from fermentation or from industrial production processes. *Water, starch sources, yeast and flavorings may be present, as in this example.

Phase B: cannabinoids and derivatives thereof, in this case, cannabidiol derived from industrial hemp, are obtained and mixed with a sufficient quantity of emulsifier. The cannabinoid-emulsifier mixture is then mixed into the alcohol mixture.

Stirring is recommended, and either a propeller or a shaking system can give the proper mixing required to disperse the cannabinoids and derivatives thereof in the body of the alcohol composition.

Hereafter, the alcohol composition with cannabinoids and derivatives thereof with a mass at about 330g was prepared.

### Example 4

### Alcohol Composition Preparation

| | Weight | Unit | % wt/wt | Phase |
|---|---|---|---|---|
| Ethanol | 89 | g | 12.86 | A |
| Fermented grape juice* | 602.3 | g | 87.04 | A |
| Hemp oil with | 0.7 | g | 0.10 | B |
| CBD at 15% | | | | |
| (wt/wt) | | | | |
| Emulsifier (gum | 3.5 | | 0.51 | B |
| acacia) | | | | |
| Total | 692 | | 100 | |

Phase A: an alcohol mixture is obtained from fermentation or from industrial production processes. *Fermented grape juice, yeast and sugars may be present, as in this example.

Phase B: cannabinoids and derivatives thereof, in this case, cannabidiol derived from industrial hemp, are obtained and mixed with a sufficient quantity of emulsifier. The cannabinoid-emulsifier mixture is then mixed into the alcohol mixture.

Stirring is recommended, and either a propeller or a shaking system can give the proper mixing required to disperse the cannabinoids and derivatives thereof in the body of the alcohol composition.

Hereafter, the alcohol composition with cannabinoids and derivatives thereof with a mass at about 731.625 g (which is equivalent to a 750 mL bottle of red wine) was prepared.

### Example 5

### Alcohol Composition Preparation

| | Weight | Unit | % wt/wt | Phase |
|---|---|---|---|---|
| Ethanol | 8 | g | 35.56 | A |
| Water* | 14.2 | g | 63.11 | A |
| Hemp oil with | 0.3 | g | 1.33 | B |
| CBD at 15% | | | | |
| (wt/wt) | | | | |
| Emulsifier (gum | 1.5 | g | 6.67 | B |
| acacia) | | | | |
| Total | 22.5 | g | 100 | |

Phase A: an alcohol mixture is obtained from fermentation or from industrial production processes. *Water with traces of flavorings may be present, as in this example.

Phase B: cannabinoids and derivatives thereof, in this case, cannabidiol derived from industrial hemp, are obtained and mixed with a sufficient quantity of emulsifier. The cannabinoids-emulsifier mixture is then mixed into the alcohol mixture.

Stirring is recommended, and either a propeller or a shaking system can give the proper mixing required to disperse the cannabinoids and derivatives thereof in the body of the alcohol composition.

Hereafter, the alcohol composition with cannabinoids and derivatives thereof with a mass at about 22.725 g was prepared.

It will be readily apparent to those skilled in the art that a number of modifications and changes may be made without departing from the spirit and the scope of the present invention. It is to be understood that any ranges, ratios, and range of ratios that can be derived from any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art will appreciate that such values are unambiguously derivative from the data presented herein.

We claim:

1. An alcoholic beverage composition comprising:
cannabinoids and derivatives thereof in the amount of about 0.001% to about 1% (wt/wt) of the composition;
an emulsifier;
ethanol present in an amount of about 2% to about 45% (wt/wt) of the compositions; and
water and other fermentation by products to total 100% by weight.

2. The alcoholic beverage of claim 1, wherein the cannabinoids and derivatives thereof are present in an amount of 0.005% to about 0.01% (wt/wt) of the composition.

3. The alcoholic beverage of claim 1, wherein the cannabinoids and derivatives thereof are present in an amount of about 0.01% to about 0.07% (wt/wt) of the composition.

4. The alcoholic beverage of claim 1, wherein the cannabinoids are present in the amount of about 0.13% to about 0.18% (wt/wt) of the composition.

5. The alcoholic beverage of claim 1, wherein ethanol is present in the amount of about 2% to about 6%.

6. The alcoholic beverage of claim 1, wherein ethanol is present in the amount of about 6% to about 15%.

7. The alcoholic beverage of claim 1, wherein ethanol is present in the amount of about 15% to about 45%

8. The alcoholic beverage of claim 1, wherein the emulsifier is chosen from the list of alginic acid, sodium alginate, potassium alginate, ammonium alginate, calcium alginate, propane-1,2-diol alginate, agar, carrageenan, locust bean gum, guar gum, tragacanth, gum acacia, xanthan gum, sorbitol, mannitol, glycerol, lecithin, pectin, amidated pectin, sodium and potassium phosphates, sodium and potassium polyphosphates, microcrystalline, methylcellulose, hydroxypropylcellulose, hydroxypropyl-methylcellulose, ethylmethylcellulose, carboxymethylcellulose, sodium, potassium, and calcium salts of fatty acids, mono- and di-glycerides of fatty acids, esters of mono- and di-glycerides of fatty acids, sucrose esters of fatty acids, sucroglycerides, polyglycerol esters of fatty acids, propane-1,2-diol esters of fatty acids, sodium stearoyl-2-lactylate, calcium stearoyl-2-lactylate, stearyl tartrate, propylene glycol alginate, and polyethylene glycol 400.

9. The alcoholic beverage of claim 8, further comprising a flavor enhancer.

10. The alcoholic beverage of claim 9, wherein the flavor enhancer is selected from the flavor enhancer group comprising of cinnamon, cucumber, mint, orange, lime, citrus, cookie dough, chocolate, vanilla, jasmine, lychee, almond, banana, grape, pear, pineapple, pine, oak, apple, pumpkin, grapefruit, watermelon, cotton sugar, durian, longan, taro, sapote, toffee nut, caramel, lotus, mango, mangosteen, coconut, coffee, strawberry, passion fruit, blueberry, raspberry, kiwi, walnut, cocoa, cherimoya, custard apple, papaya, fig, plum, nectarine, peaches, guava, honeydew, jackfruit, kumquat, loquat, palm, pomelo, persimmon, quince, and tamarind.

11. The alcoholic beverage of claim 9, further comprising a sweetener.

12. The alcoholic beverage of claim 11, wherein the sweetener is selected from the group of sucrose, maltose, fructose, sugar alcohols, xylitol, sorbitol, isomalt, artificial sweeteners, aspartame, sucralose, acesulfame potassium, saccharin.

13. The alcoholic beverage of claim 11, further comprising gaseous carbon dioxide.

14. The alcoholic beverage of claim 13, wherein gaseous carbon dioxide is present in the amount of about 2% to about 3.5% v/v.

15. The alcoholic beverage of any of claims 1, wherein the alcoholic beverage is selected from the alcoholic beverage group comprising of beer, ale, bourbon, whisky, scotch, moonshine, soju, sake, cognac, liqueurs, pure-grade alcohol, wine, tequila, gin, brandy, cider, ale, cauim, chichi, huangjiu, kasiri, kilju, kumis, mead, nihamanchi, palm wine, pulque, parakari, sakura, sonti, tepache, tonto, tiswin, maotai, akvavit, applejack, arak, awamori, baijiu, borovicka, cachaca, horilka, kaoliang, metaxa, mexcal, neutral grain spirit, ogogoro, ouzo, palinka, pisco, poitin, raki, and rakia.

16. A method to make an alcoholic beverage composition with cannabinoids and derivatives thereof comprising the steps of:
brewing an alcoholic beverage using a fermentation process;
adding a quantity of cannabinoids and derivatives thereof during the fermentation process;
adding a quantity of emulsifier; and
homogenize the composition.

17. The method of claim 16, wherein the fermentation process comprises the steps of:
milling, mashing, lautering, boiling, whirpooling, fermenting, maturing, filtering, and packaging.

18. The method of claim 17, wherein the cannabinoids and derivatives thereof are added during the fermenting step in the fermentation process.

19. The method of claim 17, wherein the cannabinoids and derivatives thereof are added during the maturing step in the fermentation process.

20. The method of claim 17, wherein the cannabinoids and derivatives thereof are added before the packaging step.

21. The method of claim 16, wherein the fermentation process comprises the steps of:
crushing the grapes; pressing the juice; fermentation; clarification; stabilization; and
storage.

22. The method of claim 21, wherein the cannabinoids and derivatives thereof are added during the fermentation step.

23. The method of claim 21, wherein the cannabinoids and derivatives thereof are added during the clarification step.

24. The method of claim 21, wherein the cannabinoids and derivatives thereof are added during the stabilization step.

25. The method of claim 21, wherein the cannabinoids and derivatives thereof are added before the storage step.

26. The method of claim 15, wherein the fermentation process comprises the steps of:
mashing, fermenting, washing, distilling, filtering, and packaging.

27. The method of claim 26, wherein the cannabinoids and derivatives thereof are added before the step of packaging.

28. A method to minimize alcohol adverse effects in a mammal comprising the steps of:
providing an alcoholic beverage comprising:
cannabinoids and derivatives thereof in an amount of about 0.001% to about 0.100% (wt/wt) of the composition;
an emulsifier;
ethanol present in an amount of about 2% to about 45% (wt/wt) of the composition;
water and other fermentation by products in a quantity sufficient for the composition to total 100%; and
consuming the alcoholic beverage at a safe level; and
wherein the adverse effects are selected from the group comprising of nausea, lethargy, muscle pain, thirst, headache, vomiting, stomach pain, decreased sleep, sensitivity to light and sound, dizziness, rapid heartbeat, red eyes, shakiness, decreased ability to concentrate, mood disturbance, depression, anxiety, irritability, unconsciousness, fatty liver, alcoholic hepatitis, and cirrhosis.

29. The method of claim 28, wherein the alcoholic beverage further comprises a flavor enhancer.

30. The method of claim 29, wherein the alcoholic beverage further comprises a sweetener.

31. The method of claim 30, wherein the alcoholic beverage further comprises gaseous carbon dioxide.
